(19) United States
(12) Patent Application Publication
(10) Pub. No.: US 2013/0196372 A1
(43) Pub. Date: Aug. 1, 2013

(54) DEVICE AND METHOD FOR CELL FREE ANALYTICAL AND PREPARATIVE PROTEIN SYNTHESIS

(71) Applicant: Ionovation GmbH, Osnabruck (DE)

(72) Inventors: Reiner Peters, Muenster (DE); Richard Wagner, Bad Iburg (DE); Richard Zimmermann, Homburg (DE)

(73) Assignee: Ionovation GmbH, Osnabruck (DE)

(21) Appl. No.: 13/669,110

(22) Filed: Nov. 5, 2012

Related U.S. Application Data
(63) Continuation of application No. 11/909,600, filed on Jun. 24, 2008, now abandoned.

(30) Foreign Application Priority Data
Mar. 24, 2005 (DE) 10 2005 013 608.7

(51) Int. Cl. C12M 1/40; C12P 21/00

(52) U.S. Cl. CPC C12M 21/18; C12P 21/00
USPC 435/68.1; 435/289.1; 435/297.1

(57) ABSTRACT

A device is disclosed which contains one or more pores 10. The pores 10 in turn contain one or more translocase proteins which from a translocation system 20.

At the pores 10, two zones, the cis zone 50 and the trans zone 60, are separated from one another on a support body 90 by means of translocation systems 20 in such a way that only those proteins that are recognized by the translocation systems 20 due to specific molecular signals can exclusively pass over from the cis zone 50 into the trans zone 60.

### CROSS REFERENCE TO RELATED APPLICATIONS

This application is a continuation under 35 U.S.C. §120 of U.S. patent application Ser. No. 11/909,600, which was filed under the provisions of 35 U.S.C. §371 based on International Application PCT/EP2006/002283 filed Mar. 13, 2006, claiming priority of German Patent Application 10 2005 013 608.7 filed Mar. 24, 2005. The disclosures of all of the foregoing applications are hereby incorporated herein by reference in their respective entireties, for all purposes.

### FIELD OF THE INVENTION

The invention relates to the field of biotechnology and molecular biology. It pertains to a process and device for the cell-free synthesis of polypeptides and proteins.

### BACKGROUND OF THE INVENTION

Peptides, polypeptides and proteins (subsumed under the term “proteins” in the following) are a class of substances playing a key role in biochemistry, molecular biology and biotechnology. For proteins, a distinction is made between soluble globular proteins and insoluble membrane proteins. The preparation of pharmacologically active globular proteins that may be employed for therapeutical purposes is an important field of activity in the biotechnological industry. Globular proteins are also put to use in diagnostic and analytical methods, for example, in the detection of pathogens. Membrane proteins are integral components of cellular membranes and mediate the transmission of signals and the transport of substances across cellular membranes. Defects in membrane proteins are the cause of many diseases. Research into the structure, function and pharmacological susceptibility of membrane proteins is an important topic of the pharmacological industry.

In the living organism, proteins are produced by a process referred to as translation in the cells' cytoplasm. During translation, the basic building blocks of the proteins, the amino acids, are gathered on ribosomes and interconnected by peptide links to form long chains The order of the amino acids in the proteins is determined by the messenger RNA (mRNA). By introducing genes into cells, the synthesis of specific mRNA species and thus specific proteins can be induced artificially.

Protein synthesis may also be performed outside the cell. Thus, the high-molecular weight components required for protein synthesis are obtained from cells by preparing cytosolic cell extracts, and these cytosolic extracts are enriched with the additionally required low-molecular weight components (for example, amino acids or high-energy triphosphates, such as ATP and GTP). In the beginning, only those proteins which were encoded by the mRNA of the starting cells (endogenous RNA) could be produced in this way. For a number of in vitro translation systems (i.e., complete systems for the cell-free protein synthesis), methods have been found for replacing the endogenous mRNA by exogenous mRNA and thus preparing specific proteins. The starting cells for these translation systems are Escherichia coli, wheat germs and rabbit reticulocytes, different methods being employed for removing the endogenous mRNA.

As compared with the methods of protein synthesis that are based on intact cells, the cell-free protein synthesis has a number of advantages, especially relating to the production speed of specific proteins and the throughput and flexibility of the processes and devices. The very preparation of cytotoxins is rendered possible by the cell-free methods in the first place. The efficient labeling of proteins for nuclear magnetic resonance and X-ray studies with specific isotopes is facilitated.

In the cell-free methods of protein synthesis, a distinction is made between static batch systems and systems that can be operated continuously. In batch systems, the preparation of the proteins takes place in a closed volume in which all high- and low-molecular weight components required for protein synthesis are present. The production of by-products and the consumption of the starting materials, such as amino acids, causes the reaction to subside. In continuously operated systems, the starting substances, also referred to as reaction educts, and the reaction products are continuously supplied to and removed from the system, respectively. There are also combined systems in which only the supply of educts is performed, for example.

In the European Patent Application EP 1 316 617, a combined system is described. In wells, for example, in a titration plate, a reaction phase is covered by a layer of supply phase, from which the reaction educts can enter the reaction phase by diffusion. Because of the layer structure, the supply phase can be renewed.

Continuous systems for cell-free protein synthesis are commercially available, such as those described, for example, the European Patent Application EP 1 061 128 or in U.S. Pat. No. 6,670,173. The systems comprise a supply chamber and a reaction chamber, separated by a semipermeable membrane. The supply chamber contains all the components that are consumed during the protein synthesis in the reaction chamber and must be replenished. These are low-molecular weight substances, such as amino acids, which can permeate the semipermeable membrane. The higher-molecular weight materials, such as the synthesized proteins and the components of the translation apparatus (e.g., ribosomes), are within the reaction chamber and cannot pass the membrane. If the exchange of components between the reaction and supply chambers takes place only passively by diffusion, the system is referred to as a continuous-exchange system. In such systems, dialysis membranes are employed as semipermeable membranes. In continuous-flow systems, the solutions in the supply and reaction chambers are constantly replaced. Due to the pressure exerted on the membrane, an increased membrane stability must be ensured for continuous-flow systems.

The existing systems for cell-free protein synthesis also have a number of disadvantages. The efficiency with which proteins of the desired functionality are prepared is low. Residues of endogenous mRNA in the cytosolic extract employed produce undesirable proteins. Exclusively soluble proteins, but not membrane proteins, can be produced. On the reaction side, the various components of the translation apparatus are present in addition to the proteins produced. Following the synthesis, the proteins must be purified. In systems with membranes, problems due to membrane clogging as well as problems of durability and stability of the membrane occur.

The purification of proteins is a tedious process in which methods of filtration, centrifugation and chromatography must be employed. A major part of the production cost for biotechnologically prepared proteins is accounted for by purification.

### SUMMARY

It is the object of the invention to prepare specific proteins of high purity.

It is a further object of the invention to simplify the analysis and/or screening of specific proteins.

These objects relate to soluble proteins, in particular, as well as membrane proteins.

These and other objects of the invention are achieved by a device comprising one or more pores. The pores in turn contain one or more translocase proteins. The translocase proteins employed form nanoscopic channels which, depending on their origin, transport non-folded, partially folded or completely folded proteins. The protein-transporting system formed by translocase proteins is referred to as translocation system in the following.

The device integrates translocation systems, as occur in biological cells or can be selectively prepared by methods of genetic engineering, into artificial systems and thus employs them for the preparation or purification of proteins.

At the pores, two zones, the cis zone and the trans zone, are separated by means of translocation systems in such a way that only those proteins that are recognized by the translocation systems due to specific molecular signals can exclusively pass over from the cis zone into the trans zone.

Due to the separation of the device into the cis zone and the trans zone, the substances necessary for the preparation of the proteins can be supplied in the cis zone. After the synthesis of the proteins and the transport thereof to the trans zone, the proteins produced are available in the trans zone.

If pores are used whose cross-sectional area is smaller than that of the translocation system, the separation into the cis zone and the trans zone can be effected by the translocation system alone.

By employing the Sec61 complex, which is a key component of protein translocase of the endoplasmic reticulum of mammals, as the translocation system, soluble eukaryotic proteins can be prepared. By adding a suitable translation system to the cis zone, the synthesis of proteins, the coupling of the protein-synthesizing ribosomes to the Sec61 complex and the translocation of the proteins into the trans zone during the synthesis thereof are induced.

If pores are used whose cross-sectional area is larger than that of the translocation system, the pores can be separated into the cis zone and trans zone by bimolecular lipid membranes or other membranes.

After the Sec61 complex has been integrated into the bimolecular lipid membranes, either the synthesis of membrane proteins and the incorporation thereof into the bimolecular lipid membranes, or the synthesis of soluble proteins and the release thereof into the trans zone can be induced, depending on the translation system employed.

The incorporation of the membrane proteins into the bimolecular lipid membranes enables an analysis of the proteins prepared at the site of synthesis, for example, by methods of optical microscopy.

The trans zone can be designed to form a closed container in which soluble proteins produced can accumulate and are available for further examination.

The trans region may also have such a design that a continuous-flow system is enabled in which new starting materials are continuously supplied in the cis zone, and the proteins prepared are discharged in the trans zone.

The pores are contained in a support body. The material of the support body may include plastic, metal, ceramic, glass or silicon depending on the intended use.

If, for pores closed on one side, the support body is at least partially made of a transparent material, then optical examinations, especially optical microscope examinations, can be performed with the contents of the pores.

In order to enable or improve the adhesion of the translocase proteins and/or the membranes to the support body, one or more layers can be applied to the support body, for example, a gold layer can be applied to the support body and covered by a lipid layer.

In addition to the proteins prepared, the solution of the trans zone only contains chaperones (folding catalysts), which may need to be separated from the proteins prepared.

The device can have such a structure that the support body separates a first chamber with the cis zones from a second chamber with the trans zones. This arrangement enables the preparation of proteins in which substances can be supplied to the first chamber or removed from the first chamber continuously or discontinuously (batch mode), or in which substances can be removed from the second chamber or supplied to the second chamber continuously or discontinuously.

When the device is used, ribosomes and nucleic acids in the first chamber can produce proteins which are then available in the second chamber.

By adding a translation system for the production of globular proteins of the endoplasmic reticulum to the cis zone, a process is obtained which can provide specific proteins of high purity in the trans zone.

By adding a translation system for producing membrane proteins to the cis zone, a process for the preparation of membrane proteins is obtained. The membrane proteins are incorporated into the bimolecular lipid membranes and can be analyzed there.

If membrane proteins are prepared, they can be examined in vitro within the membrane, for example, with respect to their transport properties for substances that are supplied in the cis zone and permeate into the trans zone.

### DESCRIPTION OF THE DRAWINGS

FIG. 1a provides an illustration of a translocation system of the invention; FIG. 1b provides an illustration of a translocation system arranged for the preparation of membrane proteins with the produced proteins incorporated into the membrane; FIG. 1c provides an illustration of a translocation system arranged for the preparation of soluble globular proteins with the produced proteins conveyed across the membrane.

FIG. 2a shows a pore separated by a translocation system and FIG. 2b shows a pore separated by a membrane into which a translocation system has been integrated.

FIGS. 3a to e show the preparation process for the separation of a pore open on both sides into cis and trans zones, where FIG. 3a illustrates the support body comprising sheets; FIG. 3b illustrates the sheets coated with a metal layer; FIG. 3c illustrates the sheets following formation of a self-assembled negatively charged monomolecular (SAM) layer on the metal layer; FIG. 3d illustrates flow of an aqueous solution over the support body such that solubilized complexes remain adhered at the entries of the pores; and FIG. 3e illustrates an alternative to FIG. 3d, including reconstitution of the complex with positively charged lipids, which bind to the SAM and spontaneously form membranes that span the pores.

FIGS. 4a to d show the preparation process for the separation of a pore open on one side into cis and trans zones, where FIG. 4a shows pores formed in the support body; FIG. 4b shows the pores closed on one side by use of a bipolar lipid membrane; FIG. 4c shows covering of the closed pores of FIG. 4b covered with a thin layer of a suitable lipid solution; and FIG. 4d and FIG. 4e show two different processes for different pore sizes.

FIG. 5 illustrates a sheet with closed pores useful for protein production.

### DETAILED DESCRIPTION OF THE INVENTION

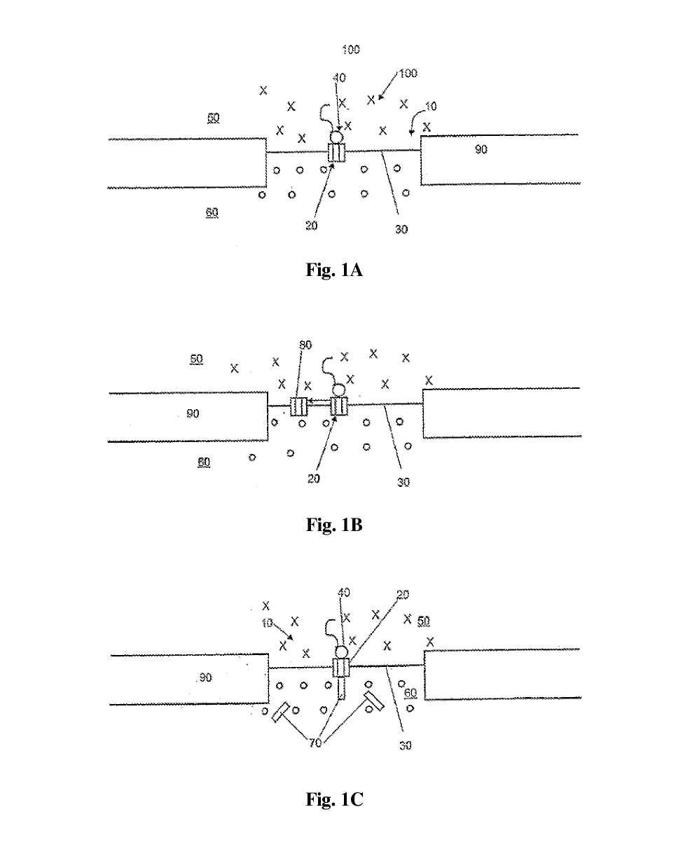

The basis of the invention is a pore 10 as shown in FIG. 1 which is closed by a translocation system 20. The translocation system 20 can be incorporated into a bimolecular lipid membrane 30. The translocation system 20 may be coupled with a translation system 40 by which specific proteins 70 can be prepared. By the translocation system 20, the proteins 70 produced are either incorporated into the membrane 30 as shown in FIG. 1a or conveyed across the membrane as shown in FIG. 1b. One example of a combination of translation system 40 and translocation system 20 is the complex consisting of ribosomes in the act of beginning with the synthesis of a protein (ribosome/nascent chain complex) and the Sec61 complex. The correct folding of the newly synthesized proteins 70 requires chaperones 60. The translocation can be effected either during the synthesis, i.e., cotranslationally, as shown in FIG. 1, or after completion of the synthesis, i.e., posttranslationally. In this case, the finished protein molecule binds to the Sec61 complex and is transported through.

FIG. 1a shows how the step of translocation is performed only partially while a membrane protein 80 is incorporated into the membrane 30. During the translocation process, the membrane protein 80 is transferred sideways from the Sec61 complex 100 into the membrane 30. For the preparation of the translation system 40, the methods that have already been developed for cell-free protein synthesis are available. In the invention, the translocation system 20 is attached to the pore 10 to block the passage through the pore 10. Thus, a separation into a cis zone 50 and a trans zone 60 is effected at the pore 10. The separation at the pore can be brought about in different ways.

Since the pore 10 is separated into the cis zone 50 and the trans zone 60, the translocation system 20 may also be used for filtrating pre-proteins, i.e., proteins containing signal sequences, recognized by the translocation system 20. This requires a driving force. An electric voltage difference, an ATP-dependent motor or an ATP-dependent turnstile may serve as the driving force. The pre-proteins are added in the cis zone 50 of pore 10, and only specific proteins can pass through the translocation system 20 into the trans zone 60. The specific proteins can be recognized by molecular signals.

The pores 10 are contained in a support body 90. The material of the support body may include plastic, metal, ceramic, glass or silicon depending on the intended use. A perforated sheet is an example of a suitable support body 90.

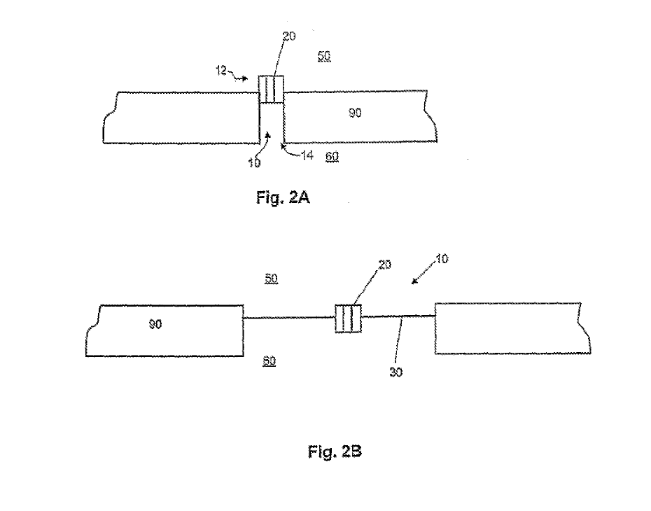

FIG. 2 shows two possibilities for the separation into the cis zone 50 and the trans zone 60 at the pore 10. FIG. 2a shows the case in which the pore 10 is smaller than the translocation system 20. Then, as shown in FIG. 2a, it is sufficient to apply the translocation system 20 in the pore 10 or at the entry 12 or exit 14 of the pore 10. For example, methods are mentioned for the applying of the Sec61 apparatus in which the pore 10 is closed with the Sec61p complex 100.

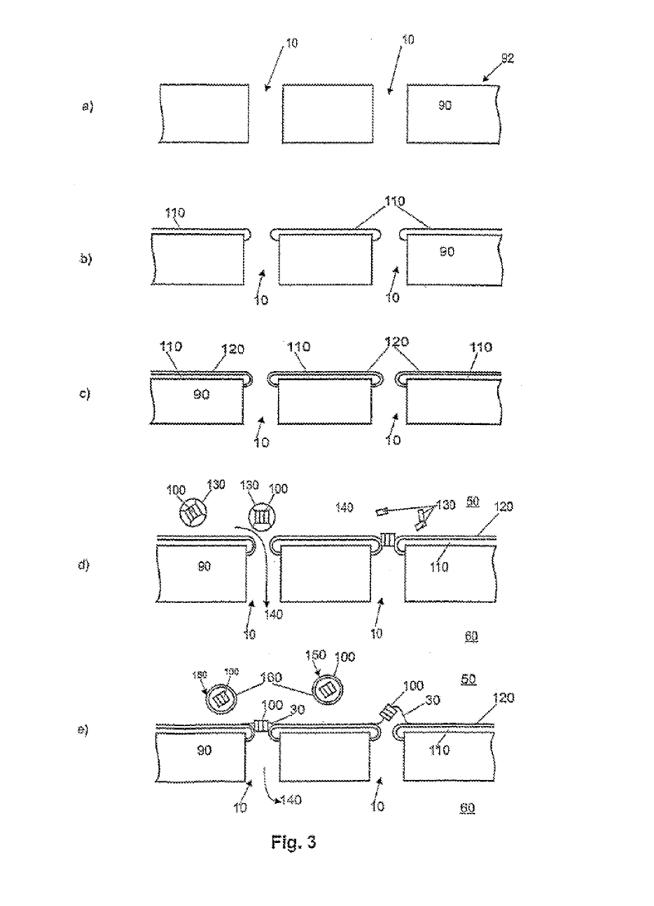

FIGS. 3a to e show the introduction of the Sec61p complex 100 into the pores 10. The diameter of the Sec61p complex 100 is about 10 nm. The pores 10 should have a size of about 15 nm. Sheets may serve as the support body 90. Sheets with a suitable pore size can be prepared, for example, by a two-step anodization of aluminum foils (H. Masuda and K. Fukada, Ordered metal nanohole arrays made by a two-step replication of honeycomb structures of anodic alumina. Science 268, 1466-1468, 1995) and are commercially available (Whatman—“Anopore Inorganic Membranes”). Other materials which have suitable pores 10 or in which pores 10 of the desired size can be generated may also be contemplated.

FIG. 3b shows how the perforated sheet 90 is coated with a metal layer 110, preferably by vapor deposition of a thin gold layer, in order to be able later to incorporate the Sec61p complex 100 into the pores 10 of the perforated sheets 90.

Immersion of the gold-deposited perforated sheet 90 with a negatively charged mercaptolipid that will covalently react with gold produces a self-assembled negatively charged monomolecular (SAM) layer 120 on a surface 92 of the perforated sheet 90, as shown in FIG. 3c. FIG. 3d shows how the support body 90 is flowed through by an aqueous solution 140 of the Sec61 complex 100 solubilized by detergents 130, so that the solubilized Sec61p complexes 100 remain adhered at the entries 12 of the pores 10 and are matched into the pores 10 by the contact between SAM 120 and detergent 130. Alternatively, as shown in FIG. 3e, the Sec61 complex 100 may also be reconstituted with positively charged lipids 160 in liposomes 150. A suspension of reconstituted liposomes 150 can be applied to the SAM 120 of the perforated sheet 90. The positively charged lipids 160 of the liposomes strongly bind to the SAM 120 on the surface 92 and spontaneously form membranes that span the pores 10 of the perforated sheet 90 and contain the Sec61p complex 100.

Pores 10 closed on one side can be realized in support bodies 90 made of different materials. Preferably, the material closing the pores is optically transparent in order to enable microscopic examinations on the proteins in the trans zone, for example. Transparency can be achieved by a very low thickness of the material employed or its optical properties. Sheets or other work pieces produced from plastic, e.g., polycarbonate, anodized aluminum or other metals, glass or glass-like solids or silicon may be used.

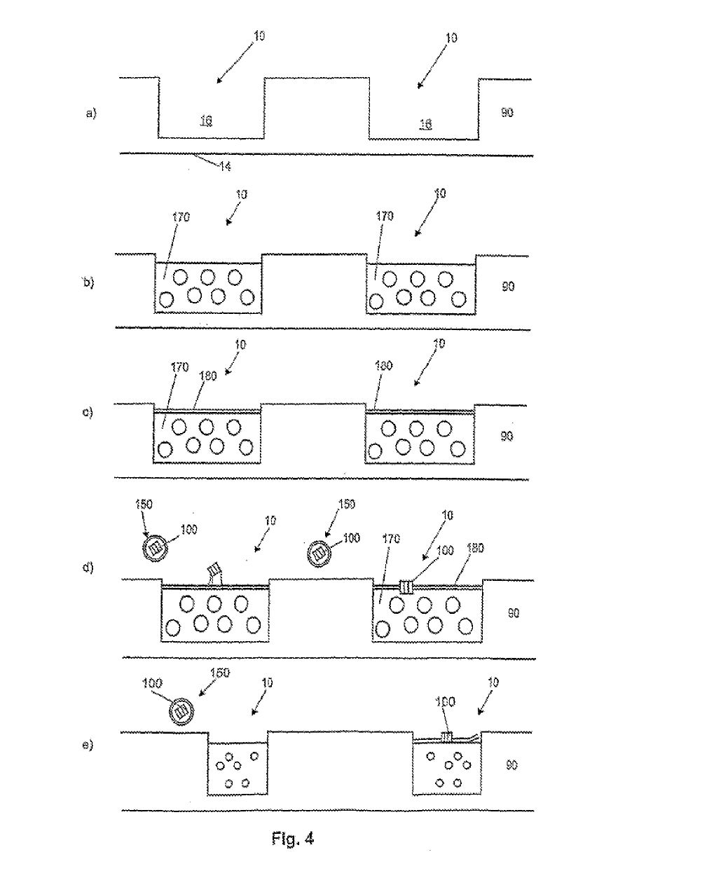

FIG. 4 shows the application of the translocation system 20 to the pores 10. In this embodiment, the pores 10 are closed at the “exit” 14′. As shown in FIG. 4a, the pores 10 are formed as recesses 16 in the support body 90, the recesses 16 mostly having diameters of from 50 nm to 100 μm for depths of from 0.1 μm to 100 μm. The dimensions of the pores 10 can be adapted to the intended use.

The pores 10 closed on one side may also be prepared by applying a perforated sheet 90 to a support. The perforated sheet 90 from FIG. 4 need not be identical with perforated sheet 90 from FIG. 3. So-called track-etched filters may be adhered to cover slips, for example. Alternatively, the pores 10 closed on one side can be prepared directly by the per se known techniques of micro- and nanostructuring. The arrangement of pores 10 may be random or regular. The area density of the pores may be from 1 per support body to 10¹²/m².

Preferably, the structure of the pores 10 is such that the trans zone 60 can be observed by an optical microscope with commercially available objectives through the closed side 14′.

In order to close pores with a bipolar lipid membrane that contains the Sec61 complex, a support body 90 with pores 10 closed on one side is covered by a physiological buffer solution 170, cf. FIG. 4. The support body 90 covered with buffer solution 170 is then covered by a thin layer of a suitable lipid solution, cf. FIG. 4c. The portions of the lipid layer that span the pores spontaneously thin out into bimolecular lipid membrane (BLM) 180. By adding a suspension of reconstituted liposomes 150 containing the Sec61p complex 100, the fusion of the reconstituted liposomes 150 with the BLMs 180 is caused, and Sec61p complexes 150 are inserted into the BLMs 180, cf. FIG. 4d. Alternatively to reconstituted liposomes, the Sec61 complex solubilized in a suitable detergent may be added to the BLM-spanned pores to insert the Sec61 complex into the BLMs. For pore diameters of up to 500 nm, the formation of the BLM 180 with the Sec61p complex 100 can be caused directly by applying the reconstituted liposomes 150 containing the Sec61p complex to the pores 10 with the formed BLM 180. This is shown in FIG. 4e. Thus, complementary ligands, such as biotin and streptavidin, should be placed in the liposome membranes and on the surface. Then, the reconstituted liposomes 150 spontaneously bind to the pores 10, open up and form the bimolecular lipid membrane 180 spanning the pore 10.

After establishing the separation into the cis zone 50 and the trans zone 60, the translation system 40 is supplied in the cis zone 50. The translation system 40 includes ribosomes that are programmed for the production of a soluble protein 70 or a membrane protein 80 depending on the intended use, and all other components required for the protein synthesis, such as amino acids, ATP. Again for illustrative purposes only, when the Sec61p complex 100 is used, the coupling of the ribosomes with the Sec61p complex 100 is initiated during the supply of the translation system 40.

Proteins 70 produced by the translation system 40 are now translocated by the translocation system 20 already during the translation process (cotranslationally) or after the completion of the translation process through the membrane 30, or incorporated into the membrane 30 by the translocation system in the case of membrane proteins 80. The translation process may be effected, for example, by the Sec619 complex 100 as the translocation system 20. The translocation process can be represented in three steps, i.e., membrane association of the precursor protein, membrane insertion and complete translocation. Aminoterminal signal peptides in the precursor proteins as well as soluble proteins of the cytosol (SRP or molecular chaperones) and a protein translocase participate in the translocation process. The heterotrimeric Sec61p complex 100 is the main component of the protein translocase. Usually, signal peptides are cleaved from the precursor protein by signal peptidases during the translocation process. The incorporation of the membrane protein 80 takes place without the translocation process being completed, and the signal peptides often remain at the membrane protein 80 and represent the transmembrane regions of the membrane protein 80. So-called tail-anchored membrane proteins 80 can be incorporated only posttranslationally. They are inserted into the membrane 30 through a carboxy-terminal end.

In order to achieve the folding of the proteins 70 into the correct tertiary structure after translocation, folding catalysts and chaperones, such as PDI and PPI, may be introduced into the solution on the cis side 50.

If the protein synthesis takes place in the pores 10 closed on one side, the produced proteins 70 accumulate in the trans zone 60 when soluble proteins 70 are prepared. After a sufficient incubation time, the proteins 70 are available for examination in the pores 10 closed on one side. A first osmotic pressure that may arise in the cis zone 50 and in the trans zone 60 due to the different concentrations of the soluble proteins 70 can be counteracted by changing the material concentrations in the cis zone 50. Materials, for example, proteins, that cannot enter the trans zone 60 may be added on the side of the cis zone 50. The addition of the materials may build up a second osmotic pressure which counteracts the first osmotic pressure.

If the trans zone 60 of the pores 10 is closed by a transparent material, examination methods of optical microscopy can be employed for examining the proteins 70 through the transparent material. When the pores 10 are closed with cover slips and applied to a slide for examination, an adaptation to the optical system of standard microscopes has already been done.

Arrangements of the pores 10 closed on one side may also be introduced into so-called microtitration plates. The arrangements are then attached at the bottom of the microchambers of the microtitration plates. For the microtitration plates with the pores 10, the known processes of automated nanoliter pipetting machines for filling are then available. The analyses of the proteins 70 prepared can be performed in parallel processes with microtitration plate readers. During the analyses, for example, fluorescence-microscopic measurements of conformational changes may then be made, and other functional parameters determined.

If membrane proteins 80 have been prepared and incorporated into the membrane 30 during the protein synthesis, these are now available for examining their properties. After the incubation time required for the preparation and insertion into the membrane 30, a substrate may be introduced, for example, into the cis zone 50, and the interaction of the substrate with the membrane proteins 80 produced can be observed. When the pores 10 are closed by a transparent material, this interaction can again be effected by methods of optical microscopy, for example, the transport of substances that are fluorescent or can be detected by a fluorescence indicator through the membrane proteins 80 can be detected by the OSTR method. When a suitable experimental set-up is employed, the dependence of the transport kinetics on electric potentials may also be examined. The measurements on membrane proteins 80 can be performed in parallelized and automated methods, as already described for soluble proteins 70, so that a high-throughput screening method for the characterization of membrane proteins 80 is thus made available.

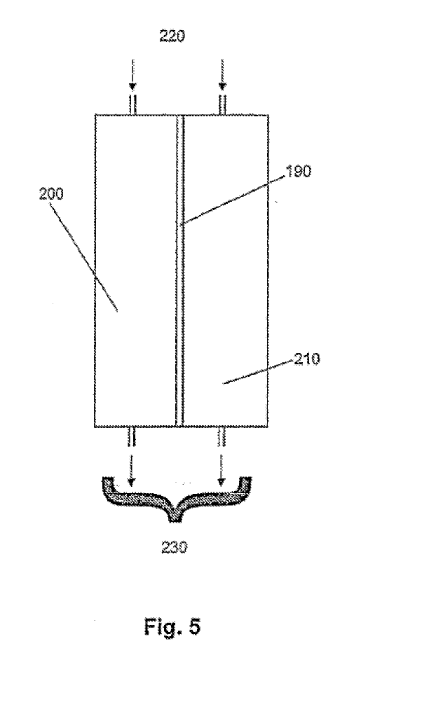

If the focus is on protein production as such rather than the analysis of proteins 70, as shown in FIG. 5, a perforated sheet 90 with the pores 10 closed by the Sec61p complex 100 as a partition wall 190 can be employed in a production device having two chambers. Into a cis chamber 200 positioned on the cis side, the substances necessary for the translation of the proteins 70 are supplied. On the trans side, there is a trans chamber 210. From the trans chamber 210, the proteins 70 produced can be removed. Both the supply of substances and the discharge of substances can be performed continuously or discontinuously by supply devices 220 and discharge devices 230, respectively.

For an efficient device that produces proteins 70 at as high a concentration as possible, a high area density of pores 10, a large surface area of the partition wall 190 with the pores 10 and a high synthetic rate are advantageous. The volume of the cis chamber 200 and that of the trans chamber 210 should be as low as possible, which results in an arrangement in which the partition wall 190 with the pores 10 has been introduced between two sheet-like borders at a low distance from the partition wall 190.

| | |
|---|---|
| 10 | pore |
| 20 | translocation system |
| 30 | membrane |
| 40 | translation system |
| 50 | cis zone |
| 60 | trans zone |
| 70 | protein produced |
| 80 | membrane protein |
| 90 | support body |
| 100 | Sec61p complex |
| 110 | metal layer |
| 120 | self-assembling monolayer |
| 130 | detergents |
| 140 | solvents |
| 150 | reconstituted liposomes |
| 160 | positively charged lipids |
| 170 | physiological buffer solution |
| 180 | bimolecular lipid membrane |
| 190 | separation plate |
| 200 | cis chamber |
| 210 | trans chamber |
| 220 | supply devices |
| 230 | discharge devices |

What is claimed is:

1. A device for the cell-free preparation of proteins, comprising one or more pores with one or more translocase proteins.

2. The device according to claim 1, wherein said one or more translocase proteins form a protein translocase.

3. The device according to claim 2, wherein a translation system is coupled to the protein translocase during use.

4. The device according to claim 1, wherein said one or more pores have a cis zone and a trans zone, the cis zone and the trans zone being separated from one another in such a way that particular proteins can pass from the cis zone into the trans zone by means of said one or more translocation proteins when in use.

5. The device according to claim 1, further comprising a membrane.

6. The device according to claim 5, wherein said one or more translocase proteins are embedded in the membrane.

7. The device according to claim 1, wherein said translocase protein is sec61p.

8. The device according to claim 5, wherein said membrane is a lipid membrane.

9. The device according to claim 10, wherein said membrane is a lipid bilayer.

10. A process for the preparation of proteins, comprising use of the device according to claim 1.

11. A device for the cell-free preparation of proteins, the device comprising:
a support body comprising a pore, wherein the pore separates a first chamber from a second chamber wherein the first chamber comprises a cis zone and the second chamber comprises a trans zone;
a translocation system spanning the pore, the translocation system comprising:
a bimolecular lipid membrane;
a translation system embedded in the membrane, the translation system comprising one or more translocase proteins wherein at least one of the translocase proteins is sec61p,
wherein the translocation system is positioned between the first chamber and the second chamber for translocation of proteins between the cis zone and the trans zone.
